(19) United States
(12) Patent Application Publication
(10) Pub. No.: US 2005/0266415 A1
(43) Pub. Date: Dec. 1, 2005

(54) Method for sampling gas-borne matter

(75) Inventor: Scott Ryan, Ocala, FL (US)

Correspondence Address: FOLEY & LARDNER, 777 EAST WISCONSIN AVENUE, SUITE 3800, MILWAUKEE, WI, 53202-5308, US

(73) Assignee: Zefon International, Inc.

(21) Appl. No.: 10/857,290

(22) Filed: May 28, 2004

(51) Int. Cl. C12Q 1/68; G01N 33/53; G01N 33/537; G01N 33/543

(52) U.S. Cl. 435/6; 435/7.92

(57) ABSTRACT

A method for sampling gas-borne matter using a collection device comprising an inlet, an outlet, and a slide provided adjacent the inlet includes drawing a gas through the collection device such that gas-borne matter is collected on the slide, sealing at least one of the inlet and the outlet, and introducing a liquid into the collection device to remove the collected gas-borne matter from the slide.

### FIELD

The present invention relates to collection devices for sampling gas-borne matter (e.g., particulate matter, bacteria, mold spores, etc.) and methods for using such collection devices.

### BACKGROUND

In a variety of environments, it may be desirable to collect and analyze matter or material present in a gaseous (e.g., air) atmosphere. For example, in a factory where materials are used that may be detrimental to human health, it may be desirable to determine the amount and types of matter present in the atmosphere so that factory workers are not exposed to unsafe or undesirable levels of airborne materials. Other environments in which air sampling may be beneficial include office buildings, houses, hospitals, clean rooms, outdoors, and others.

It is known to provide collection devices (e.g., particle impaction devices, microscope slides, Petri dishes, or other devices) for collecting and retaining gas-borne matter. In use, a gas (e.g., air) is drawn toward the collection device. Matter is collected on or in the collection device, where the matter is retained until analysis can be performed. One known type of collection device is a cassette or cartridge type sampling device, such as the Air-O-Cell brand product manufactured by the assignee of the present application, Zefon International, Inc. of Ocala, Fla.

One difficulty with the use of conventional collection devices is that the collection devices may become contaminated with continued use. For example, after sampling is complete, the collection medium (e.g., agar medium) is removed from the collection device and the collection device is cleaned. Remnants of past samples and media may remain after cleaning, which may affect results of subsequent sampling.

Some collection devices require that a user place an appropriate amount of collection medium on or in the collection device (e.g., by applying a tacky material on a plate in the collection device). Variability due to human error in the thickness and amount of collection medium may affect results of subsequent sampling. For example, overloading the collection device with collection medium may alter the gas flow characteristics of the device (e.g., the collection medium may block an air inlet) or collection efficiency may be reduced.

It would be advantageous to provide an improved collection device for use in gas sampling. It would also be advantageous to provide a collection device and/or method that allows for the relatively simple and efficient collection and removal or extraction of sampled matter from a collection device. It would also be advantageous to provide a collection device and/or a method that allows for the archival, storage, and shipping of matter sampled from a gaseous environment. It would also be desirable to provide a collection device that allows a user of the collection device to sample multiple types of gas-borne matter in a single sampling period. It would be desirable to provide a collection device and/or a method that includes one or more of these or other advantageous features.

### SUMMARY

An exemplary embodiment relates to a method for sampling gas-borne matter using a collection device comprising an inlet, an outlet, and a slide provided adjacent the inlet. The method includes drawing a gas through the collection device such that gas-borne matter is collected on the slide, sealing at least one of the inlet and the outlet, and introducing a liquid into the collection device to remove the collected gas-borne matter from the slide.

Another exemplary embodiment relates to a method for collecting gas-borne matter. The method includes providing a sampling device comprising at least one inlet and at least one outlet for allowing the passage of a gas-through the sampling device and a plate provided within the sampling device for collecting gas-borne matter. The method also includes drawing a gas through the sampling device such that the plate is impacted with gas-borne matter. The method further includes blocking at least one of the inlet and the outlet after the step of drawing a gas through the sampling device and introducing a liquid into the sampling device after blocking at least one of the inlet and the outlet. The method further includes removing at least a portion of the liquid from the sampling device.

Another exemplary embodiment relates to a method for collecting gas-borne matter. The method includes providing a collection device, the collection device including an inlet, an outlet, and a plate provided adjacent the inlet for collecting gas-borne matter. The method also includes drawing a gas through the sampling device to collect gas-borne matter on the plate, blocking at least one of the inlet and the outlet after the step of drawing a gas through the collection device, and introducing a liquid into the collection device after blocking at least one of the inlet and the outlet. The liquid is selected from the group consisting of water, peptone water, mineral oil, a surfactant, and combinations thereof. The method further includes analyzing the matter collected using at least a portion of the liquid introduced into the collection device.

### BRIEF DESCRIPTION OF THE DRAWINGS

FIG. 1 is a perspective view of an collection device according to an exemplary embodiment.

FIG. 2 is an exploded a perspective view of the collection device shown in FIG. 1.

FIG. 3 is an exploded perspective view of a portion of the collection device shown in FIG. 1.

FIG. 4 is a cross-sectional view of a portion of the collection device shown in FIG. 1, taken across line 4-4.

FIG. 5 is a cross-sectional view of a portion of the collection device shown in FIG. 1, taken across line 5-5.

FIG. 6 is a cross-sectional view of a portion of the collection device shown in FIG. 1, taken across line 6-6.

FIG. 7 is an exploded perspective view of a collection device according to another exemplary embodiment.

FIG. 8 is a cross-sectional view of a portion of the collection device shown in FIG. 6, taken across line 8-8 and showing a plate or slide provided on a ledge or rim.

FIG. 9 is a cross-sectional view of a portion of the collection device shown in FIG. 7, taken across line 9-9.

FIG. 10 is a cross-sectional view of the portion of the collection device shown in FIG. 7, showing a plate or slide provided within a trench or trough provided in the portion.

FIG. 11 is a cross-sectional view of the portion of the collection device shown in FIG. 9, taken across line 11-11.

FIG. 12 is a cross-sectional view of a collection device according to

FIG. 13 is a cross-sectional view of a collection device according to another exemplary embodiment.

FIG. 14 is a cross-sectional view of a collection device according to another exemplary embodiment.

FIG. 15 is a cross-sectional view of a collection device according to another exemplary embodiment showing the use of multiple slides or plates with a single inlet.

FIG. 16 is an exploded cross-sectional view of an assembly including a plurality collection devices according to another exemplary embodiment.

FIG. 17 is a cross-sectional view of the assembly shown in FIG. 16 showing the coupling of a plurality of collection devices coupled together.

### DETAILED DESCRIPTION OF THE EXEMPLARY EMBODIMENTS

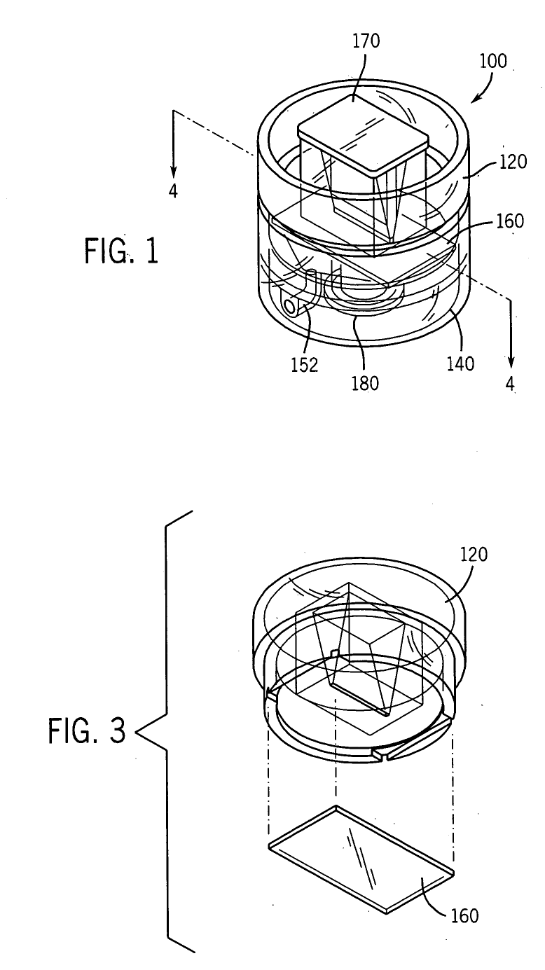

FIGS. 1-6 show a collection device or apparatus 100 according to an exemplary embodiment for use in gas-borne matter sampling. According to an exemplary embodiment, collection device 100 is configured for use with a pump or other device (hereinafter referred to as an “air sampling device” or a “gas sampling device”) used to sample a gas such as air, for example, by drawing or pulling air or another gas through the collection device using an impeller, blower fan, or other type of fan or pump (e.g., a vacuum pump). Such an air sampling device may be connected either directly or indirectly to collection device 100.

According to an exemplary embodiment, collection device 100 includes a top or upper portion 120, a bottom or lower portion 140, and a sampling plate or slide 160. Top portion 120 is selectively or removably coupled to lower portion 140. In this manner, plate 160 may be removed from collection device 100 by decoupling top portion 120 from bottom portion 140. According to another exemplary embodiment, top portion 120 may be permanently coupled to bottom portion 140 such that plate 160 may not be removed from collection device 100.

Top portion 120 and bottom portion 140 may be made from a variety of materials, including polymeric, metal, ceramic, glass, or other materials suitable for use in a collection device. According to an exemplary embodiment, top portion 120 and bottom portion 140 are made of a styrene-based copolymer (e.g., a styrene-acrylonitrile copolymer). According to various other exemplary embodiments, other materials may be used to form top portion 120 and bottom portion 140.

According to an exemplary embodiment, both top portion 120 and a bottom portion 140 are made of a relatively transparent material, such that one may observe the interior of the collection device without disassembling collection device 100. According to another exemplary embodiment, one or both of the top portion and a bottom portion may be made of a relatively opaque or translucent material. For example, its may be desirable to provide a relatively dark environment for certain matter being sampled (e.g., certain types of bacteria may require a relatively dark environment).

Collection device 100 is shown in the form of a cassette or cartridge, although various configurations for may be used according to other exemplary embodiments. According to the exemplary embodiment shown in FIG. 1, collection device 100 has a relatively cylindrical shape. According to other exemplary embodiments, other sizes and shapes for the collection device may be used. For example, according to another exemplary embodiment, a collection device may have a rectangular solid or cubic shape.

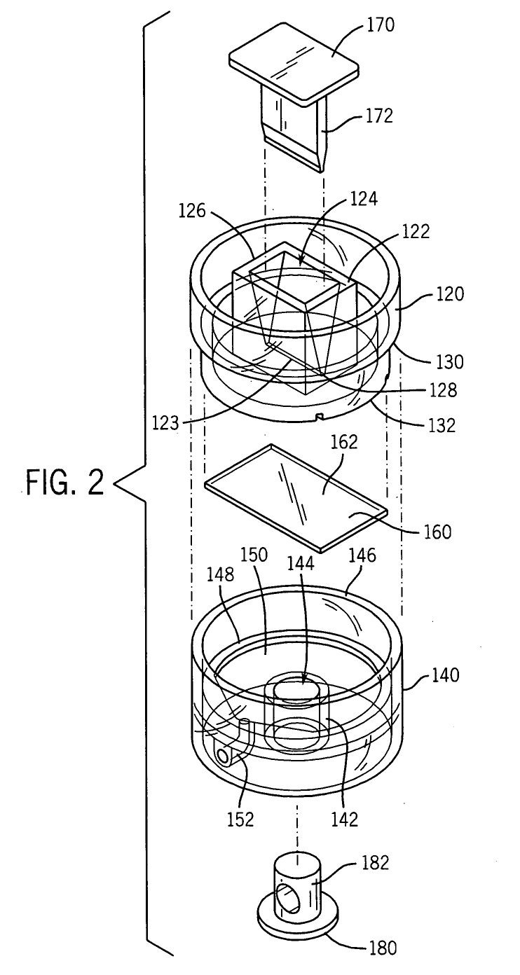

As shown in FIG. 2, top portion 120 includes an inlet 122 that defines an aperture or opening 124 through which a gas is drawn during sampling. According to an exemplary embodiment, the size of aperture 124 defined by inlet 122 narrows from a top portion 126 to a bottom portion 128 of inlet 122. Aperture 124 has a generally rectangular shape when viewed in the axial direction. The size (e.g., area) of the rectangle defined by inlet 122 decreases from top portion 126 to bottom portion 128 in a substantially continuous manner to form a slit 123 in top portion 120. According to an exemplary embodiment, the width of aperture 124 (e.g., the longer side of the rectangle) remains constant between top portion 126 and bottom portion 128 while the length (e.g., the shorter side of the rectangle) decreases with increasing distance from top portion 126 of aperture 124.

According to another exemplary embodiment, both the length and width of the rectangle forming the aperture decrease with increasing distance from the top of the inlet. According to other alternative embodiments, the shape of the inlet and/or aperture may differ. For example, an aperture may have a generally circular, square, oval, or other shape when viewed in the axial direction. Such inlets and/or apertures according to alternative embodiments may or may not decrease in area with increasing distance from the top of the inlets. For example, where an aperture and/or inlet is provided with a generally circular cross-section viewed in the axial direction, the aperture and/or inlet may resemble a funnel (e.g., the area decreases with increasing distance from the top of the inlet) or may resemble a cylinder (e.g., the area does not decrease with increasing distance from the top of the inlet). Any of a variety of shapes and configurations may be provided for the aperture and/or inlet according to various other embodiments, and the shape, size, and other characteristics may be optimized for a particular application.

Bottom portion 140 of collection device 100 includes an outlet or exit port 142 defining an aperture or opening 144 through which air is drawn during sampling. According to an exemplary embodiment, outlet 142 has a generally circular cross-sectional shape when viewed in the axial direction. Outlet 142 may taper from a larger diameter to a smaller diameter. According to various other exemplary embodiments, the size and/or shape of outlet 142 may differ. For example, according to another exemplary embodiment, the outlet may have a generally square or oval cross-sectional shape when viewed in the axial direction. Further, the outlet may or may not taper along its length according to various other exemplary embodiments.

When collection device 100 is assembled, a portion of top portion 120 is inserted within a portion of bottom portion 140 such that a first rim or surface 130 provided on top portion 120 abuts a first rim or surface 146 provided on bottom portion 140 and a second rim or surface 132 provided on top portion 20 abuts a second rim or surface 148 provided on bottom portion 140. Top portion 120 may be relatively securely coupled to bottom portion 140 by means of a friction fit. According to other exemplary embodiment, other ways of connecting the top portion to the bottom portion may be utilized (e.g., glue, fasteners such as screws and bolts, welding, etc.). Collection device 100 may be disassembled by decoupling top portion 120 and bottom portion 140. For example, top portion 120 and bottom portion 140 may be decoupled to allow removal of plate 160 after sampling has been completed.

Plate or slide 160 is provided such that it is intermediate or between inlet 122 and outlet 142. A top surface 162 of plate 160 is provided adjacent or proximate slit 123 formed in top portion 120. Matter carried in gas drawn through collection device 100 impacts top surface 162 of plate 160 such that the matter is retained on plate 160 four subsequent sampling. According to another exemplary embodiment, plate 160 may be provided such that it is not intermediate inlet 122 and outlet 142 (e.g., the plate may be provided adjacent an inlet but spaced away from an outlet, etc., such that it is not directly intermediate the inlet and the outlet).

While plate 160 is shown as having a particular shape in the accompanying FIGURES, according to other exemplary embodiments, the plate or slide may have any of a variety of sizes, shapes, and/or configurations (e.g., oval, circle, hexagon, etc.), which may be chosen based on any of a variety of factors (e.g., required size of sampling area, manufacturability, cost, etc.).

Plate 160 may be made of any suitable material, including glass, porous glass fiber filters, ceramic, porous plastic, metal (e.g., aluminum, steel, etc.), or any other suitable material (e.g., a porous rigid material). According to an exemplary embodiment, plate 160 is made of glass and has a generally rectangular shape. Plate 160 is relatively thin (e.g., between approximately 0.001 and 0.125 inches), although the thickness may vary in alternative embodiments. For example, the plate may be formed to have a thickness similar to that of conventional microscope slides. Other configurations may also be used for plate 160. For example, the plate may be made of a polymeric material and/or may be formed in the shape of an octagon, triangle, square, circle, oval, or any other suitable shape (e.g., cup-shaped, dish-shaped, etc.).

According to one exemplary embodiment, plate 160 may be a clean plate or slide (e.g., the plate may not have a substance provided thereon for capturing gas-borne matter). According to other exemplary embodiments, plate 160 may be provided with a substance configured or adapted to capture matter (e.g., the substance may have a relatively tacky characteristic that is designed to capture matter carried within a gas flowing through the collection device). For example, such a substance may be provided as a suspension medium or gel that is adapted for maintaining a viable matter in a living state without promoting growth, as disclosed in U.S. patent application Ser. No. 10/808,114 filed Mar. 24, 2004 and entitled “Gas-Borne Matter Collection Device,” the entire disclosure of which is hereby incorporated by reference.

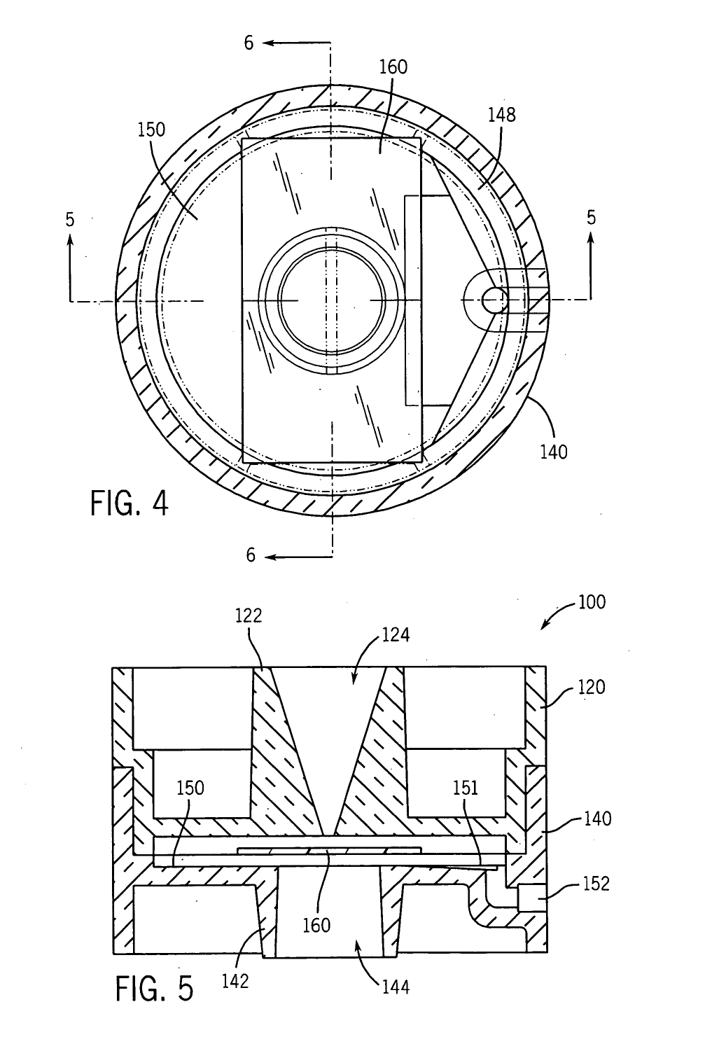

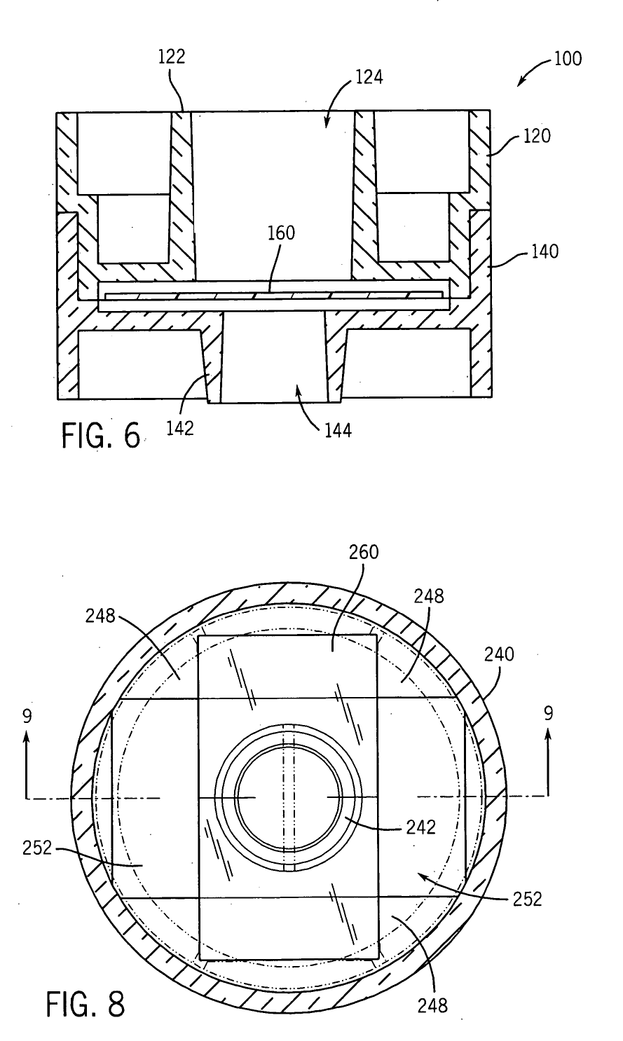

During sampling, a portion of plate 160 (e.g., the corners of plate 160, as shown in FIG. 4) rests on rim 148 of bottom portion 140 such that plate 160 is elevated above a surface 150 of bottom portion 140. FIGS. 5 and 6 show cross-sectional views of collection device 100 illustrating the arrangement of plate 160 within collection device 100. Gas flowing into collection device 100 flows through inlet 122, around plate 160, and out of collection device 100 through outlet 142. Features such as protrusions or cutouts may be provided to secure plate 160 in place (e.g., to prevent movement of plate during sampling) according to other exemplary embodiments.

Bottom portion 140 includes an aperture 152 in the form of a port or channel that is configured to allow the introduction of liquid into and the extraction or removal of liquid from collection device 100. Aperture 152 extends between the interior of collection device 100 and the exterior of collection device 100 (e.g., extending between bottom surface 150 and an exterior surface of bottom portion 140). While aperture 152 is shown as extending between surface 150 and a side external surface of collection device 100, aperture 152 may extend between surface 150 and a bottom of collection device 100 according to another exemplary embodiment. According to yet another exemplary embodiment, outlet 142 may be used for the injection and extraction of liquid from collection device 100. For example, a barrier (e.g., such as barrier 170 described below) may be inserted into inlet 124, after which liquid may be injected into collection device 100 through outlet 142. After vibration or other means are used to separate impacted material from plate 160, the liquid may then be extracted through outlet 142. According to another exemplary embodiment, inlet 122 may be used for the injection and extraction of liquid from collection device 100 (e.g., a barrier may be inserted into outlet 142, after which liquid may be injected into collection device 100 through inlet 122, followed by removal of impacted material from plate 160).

When sampling of gas-borne matter is completed, a barrier 170 may be inserted into inlet 122 to effectively seal inlet 122. Similarly, a barrier 180 may be inserted into outlets 142 to effectively seal outlet 142. Barriers 170 and 180 are configured for removable coupling to collection device 100 according to an exemplary embodiment. A portion 172 of barrier 170 has a size and shape configured for relatively tight fitment with aperture 124 of inlet 122, and a portion 182 of barrier 180 has a size and shape configured for relatively tight fitment with aperture 144 of inlet 142. By inserting barrier 170 into inlet 122 and barrier 180 into outlet 142, a relatively watertight seal is formed that allows liquid to be retained within the collection device 100.

Once barrier 170 and barrier 180 are in place, the liquid may be injected into collection device 100 through aperture 152. For example, water, peptone water, a surfactant (e.g., tween 80), mineral oil, or another liquid may be injected using a syringe or needle into collection device 100. According to an exemplary embodiment, between approximately 0.1 and 25.0 milliliters (ml) of liquid such as water may be injected into the collection device. According to another exemplary embodiment, between approximately 0.25 and 1.0 ml of liquid may be injected into the collection device. The liquid selected according to any of a variety of other exemplary embodiments may depend on any of a variety of factors, including the type of matter collected within collection device 100.

According to an exemplary embodiment, the collection device utilizes a substance provided on the plate for capturing gas-borne matter, and the liquid is selected such that the substance is soluble in the liquid.

Collection device 100 may then be vibrated, shaken or agitated to remove matter impacted upon plate 160, after which the liquid may be removed or extracted from collection device 100 through suction or other means (e.g., a syringe maybe utilized to remove the liquid from the collection device). According to another exemplary embodiment, the liquid introduced into collection device 100 may dissolve a substance provided on plate 160, such that removal of the liquid from collection device 100 will include matter impacted into the substance. Various other methods may be used to extract impacted matter from collection device 100. For example, according to an exemplary embodiment, the substance provided on plate 160 may be scraped off of the plate and analyzed or placed in a liquid to remove the impacted matter. According to another exemplary embodiment, the entire plate 160 may be removed from collection device 100 and placed into a liquid (e.g., in a test tube or Petri dish) to remove the substance and sampled matter. According to another exemplary embodiment, the plate may be rinsed with water or another liquid (e.g., mineral oil) to remove the substance and sampled matter.

Surface 150 is configured to allow liquid to escape through aperture 152 in bottom portion 140. According to an exemplary embodiment, surface 150 is angled or sloped such that liquid retained within collection device 100 flows toward aperture 152 (see, e.g., FIG. 4, which shows a sloped portion 151 of surface 150).

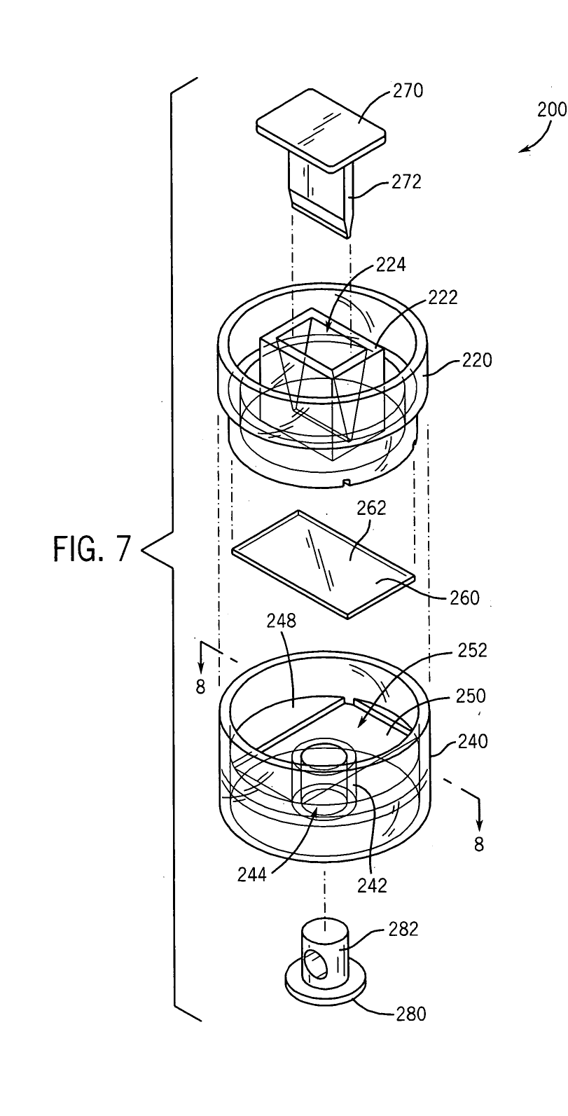

FIGS. 7-11 show a collection device 200 according to another exemplary embodiment. Collection device 200 includes a top portion 220 having an inlet 222 defining an opening 224 and a bottom portion 240 having an outlet 242 defining an opening 244. A barrier 270 having an extension 272 and a barrier 280 having an extension 282 are also provided. A slide 260 having a top surface 262 may be provided within collection device 200. Similar materials may be utilized for the components of collection device 200 as were described above with respect to collection device 100.

Top portion 220 of collection device 200 is illustrated as being similar to top portion 120 illustrated in FIG. 1 (e.g., inlet 222 is shown as having a shape similar to that of inlet 122). As shown in FIG. 7, however, bottom portion 240 does not include an aperture such as aperture 152 shown in FIG. 1.

Bottom portion 240 includes a trench or trough 252 in which plate 260 may be provided. Trench 252 is defined by a ledge or shelf 248. According to an exemplary embodiment, trench 252 has a size and shape configured to closely match that of plate 260.

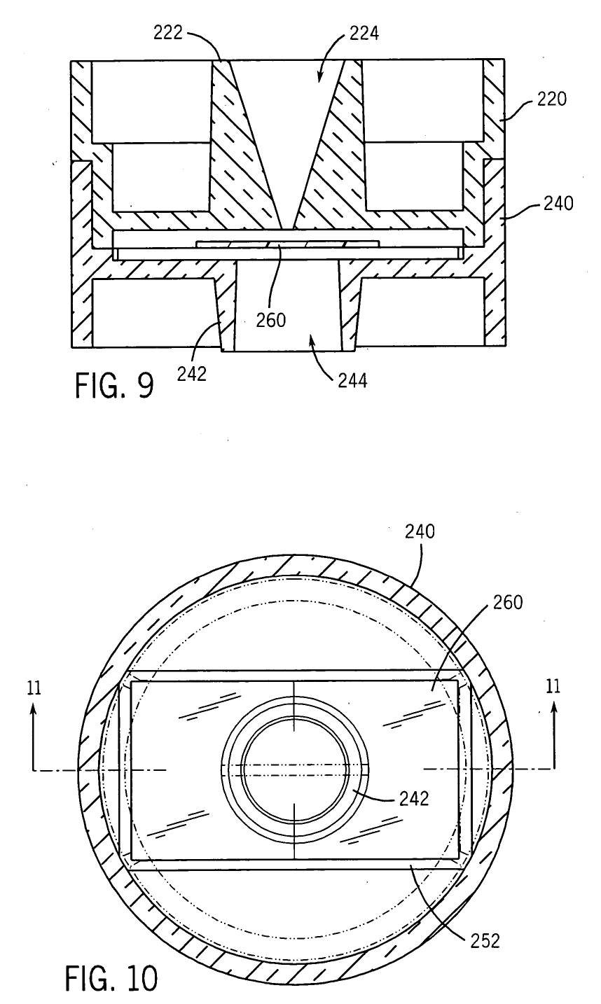

FIGS. 8-11 illustrate the positioning of the plate 260 within collection device 200 during sampling and extraction of impacted matter. During sampling of gas-borne matter, plate 260 is provided within collection device 200 such that it rests upon ledge 248. Gas entering collection device 200 flows through inlet 222, around plate 260, and out of collection device 200 through outlet 242. Such a configuration is shown in FIGS. 8-9.

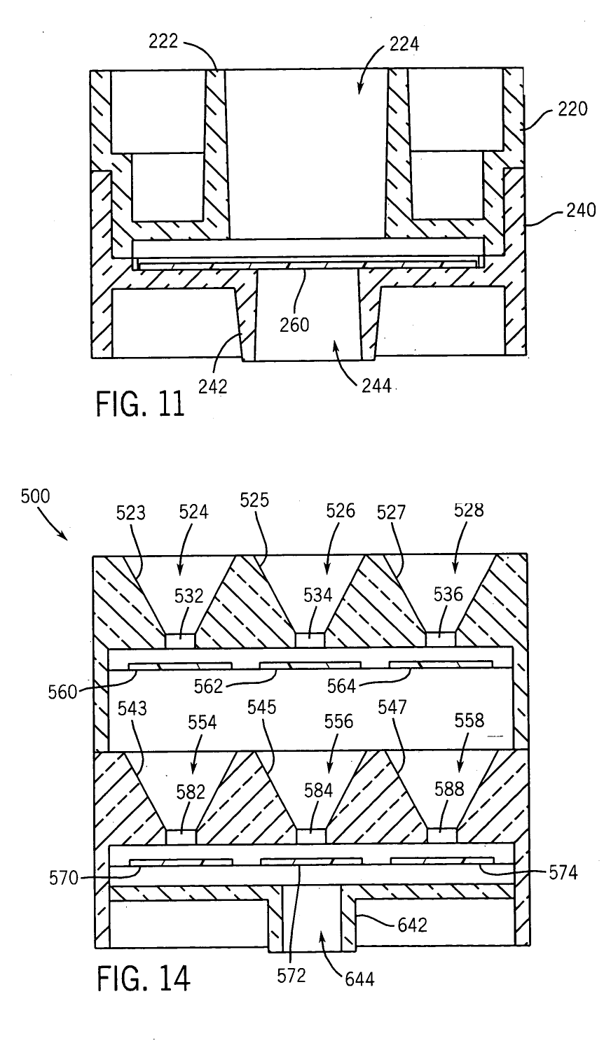

Once sampling of gas-borne matter is completed, top portion 220 is separated from bottom portion 240 and plate 260 is rotated relative to its original position and provided with trench 252 such that it rests upon a surface 250 of bottom portion 240, has shown in FIGS. 10-11. Collection device 200 may then be sealed (e.g., by inserting barriers 270 and 280 into their respective inlet and outlet, etc.) and stored or shipped for analysis. For example, collection device 200 may be shipped to a laboratory so that the impacted matter may be extracted from collection device 200. According to other exemplary embodiments, the impacted matter may be extracted from collection device 200 immediately after sampling is completed. For example, the collection device may be coupled to a vortexer or to another type of device that shakes or vibrates the collection device to remove impacted matter from the plate. According to another exemplary embodiment, the plate may be used for direct microscope analysis (e.g., by analyzing the impacted matter on the plate once the plate is removed from the collection device).

It should be noted that for applications in which water or another liquid are introduced into the collection device, all or a portion of the liquid may be frozen (either within the collection device or subsequent to removal from collection device). In this manner, sampled matter provided within the liquid may be archived or stored for later use or analysis.

It should be noted that any other of a variety of analysis techniques may be utilized with the matter collected by the collection device. For example, according to one exemplary embodiment, an Enzyme Linked Immunosorbent Assay (ELISA) analysis technique may be utilized. ELISA testing is a calorimetric test used to detect and measure antigens or antibodies in a solution. According to this embodiment, a particulate material or other matter is sampled using collection device 100. A liquid such as water is injected into collection device 100 and suspended in the liquid such that the liquid includes a target antibody. To determine the amount of antibodies in the liquid, a specific antigen coupled to an enzyme is added to the liquid (either in the collection device or after extraction from the collection device). The antigen will combine with the antibody, which causes the enzyme in the solution to change color. The depth of color change in the liquid is dependent on the amount of target antibodies present. The color of the liquid may be compared with colors shown in a chart to determine the concentration or other feature of the sampled matter and/or liquid. ELISA testing may be performed on the sample within the collection device or after extraction or removal of the sample from the collection device.

According to another exemplary embodiment, a Polymerase Chain Reaction (PCR) analysis technique may be utilized. PCR testing is a calorimetric test used to detect and measure the presence of a DNA sequence which is usually unique to a particular organism. According to this embodiment, a particulate material or other matter is sampled using collection device 100. A liquid such as water is injected into collection device 100 and suspended in the liquid such that the liquid includes a target DNA. To determine which DNA is in the liquid, a specific reagent is added to the liquid. The reagent will attach to its target DNA sequence, which causes the liquid to change color. The depth of color change in the solution is dependent on the amount of target DNA present. The color of the liquid may be compared with colors shown in a chart to determine the concentration or other feature of the sampled matter and/or liquid. PCR testing may be performed on the sample within the collection device or after extraction or removal of the sample from the collection device.

According to another exemplary embodiment, organisms present in sampled matter may be analyzed using a fluorometric or fluorescence detection method. According to this embodiment, a material including one or more types of organisms is sampled using collection device 100. A liquid such as water is introduced into the collection device, and a reagent is added to the liquid. The reagent reacts with enzymes in the sample, which causes fluorescence, after which the sample is placed in a fluorometer to measure the amount of fluorescence. Fluorescence testing may be performed on the sample within the collection device or after extraction or removal of the sample from the collection device.

While the FIGURES illustrate collection devices that include a top portion that is inserted into a bottom portion, according to another exemplary embodiment a bottom portion may be inserted into a top portion. According to another embodiment, neither of the top and bottom portions are inserted into each other, and coupling of the top portion and bottom portion is accomplished by any of a variety of other methods. According to various other embodiments, the top and bottom portions may be secured together using adhesives, ultrasonic welding or sealing, a screw-type arrangement, a snap-fit type arrangement, or any other suitable means.

According to an exemplary embodiment, the collection devices disclosed herein may be disposable or non-reusable type collection devices (i.e., the collection device may be intended as a single-use type component that is discarded after use). According to other exemplary embodiments, the collection devices may be reusable type collection devices (e.g., the plate may be cleaned and re-inserted into the collection device or the plate may be replaced with a new plate). One advantageous feature of using a disposable collection device is that cleaning of the plate is eliminated. A related advantageous feature of using a disposable collection device is that errors in sampling due to contamination of the substance that may be provided on the plate and/or to variations in application of new substance on the plate may be reduced or eliminated. According to another exemplary embodiment, plates (e.g., glass slides, etc.) may be provided as separate components for use in a collection device and sold separately from the collection device.

One or more of the components of the collection devices shown and described herein may include a marking or other identification for identifying or associating the sampled matter with a particular collection device. For example, a plate or slide such as plate 160 may include an engraving, and embossment, or an ink marking that acts as an identification marking to allow identification of the plate with a particular collection device. One or both of the top portion and bottom portion of the collection device may also include an identification marking. The identification marking is used on the top or bottom portion may be identical to that used on the plate to allow a correlation between the identification markings. While any other variety of identification markings may be utilized, according to an exemplary embodiment, the identification marking includes a serial number.

One advantageous feature of the collection devices described herein is that such collections devices may be sterilized subsequent to assembly an maintained as sterile collection devices during shipping and storage. For example, subsequent to sterilization, the inlet, outlet, and any apertures or gaps between various portions of the collection device may be sealed (e.g., using a seal such as a sticker or label, etc.).

While collection devices 100 and 200 are shown as having a single outlet and a single inlet, according to other exemplary embodiments, any number of outlets and inlets may be provided in a collection device having any of a variety of different configurations (e.g., one outlet may have a relatively circular opening while a second outlet may have a relatively rectangular opening, etc.). A sampling plate or slide may have different sampling mediums (e.g., suspension media) provided in different areas such that a different sampling medium is provided adjacent each of the plurality of inlets.

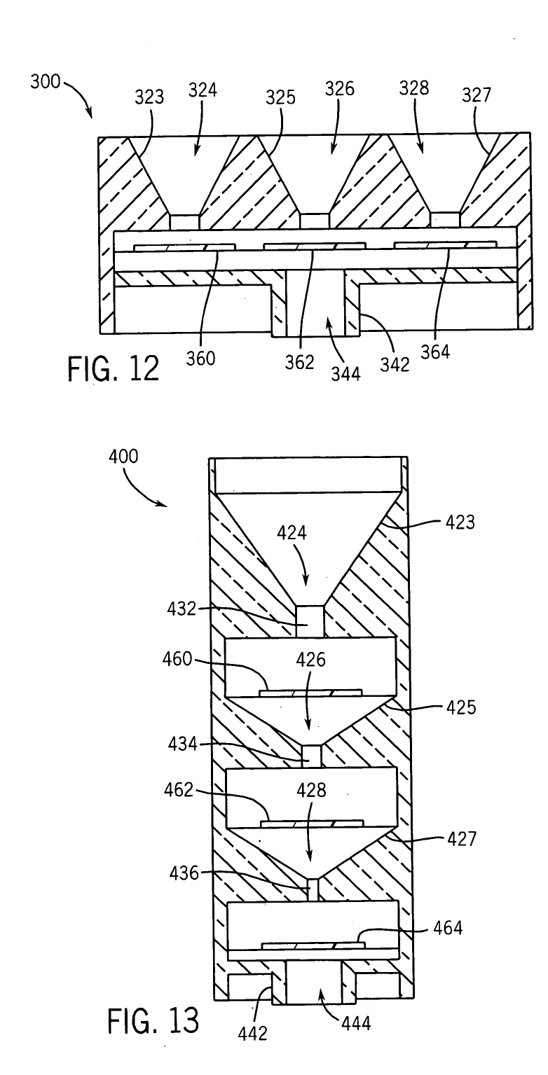

FIG. 12 is a cross-sectional view of a collection device 300 according to an exemplary embodiment. Collection device 300 includes three inlets 323, 325, and 327 that define three separate openings 324, 326, and 328. Inlets 323, 325, and 327 are arranged such that they are next to each other (as opposed to being arranged vertically, as shown in FIG. 13). Collection device 300 also includes a single outlet 342 defining an opening 344. According to other exemplary embodiments, a different number of outlets or inlets may be provided. As shown in FIG. 12, each of inlets 323, 325, and 327 is associated with a single plate or slide 360, 362. and 364. That is, plate 360 is provided beneath inlet 323, plate 362 is provided beneath inlet 325, and plate 364 is provided beneath inlet 327. Each of the plates may be used to test for different matter (e.g., plate 360 may be used to sample for bacteria, plate 362 for nanoparticles, and plate 264 for other microorganisms) or for similar matter. The plates may include similar or different collection mediums, or no collection medium at all. Each of the inlets may have an identical shape or may differ from one another (e.g., the size of the opening defined by each of the inlets may be optimized to collect particular types of matter). It should be noted that while collection device 300 is shown as having three inlets, a different number of inlets may be provided (e.g., two inlets, four or more inlets, etc.).

FIG. 13 is a cross-sectional view of a collection device 400 according to another exemplary embodiment. Collection device 400 includes three inlets 423, 425, and 427 that define three separate openings 424, 426, and 428. Inlets 423, 425, and 427 are arranged such that they are stacked vertically. Collection device 400 also includes a single outlet 442 defining an opening 444.

Each of inlets 423, 425, and 427 is associated with a single plate or slide 460, 462. and 464. Each of the plates may be used to test for different or similar matter. The plates may include similar or different collection mediums, or no collection medium at all. Each of the inlets may have an identical shape or may differ from one another (e.g., the size of the opening defined by each of the inlets may be optimized to collect particular types of matter). As shown in FIG. 13, inlets 423, 425, and 427 include openings 432, 434, and 436 that decrease in size from the top to the bottom of the collection device. It should be noted that while collection device 400 is shown as having three inlets, a different number of inlets may be provided (e.g., two inlets, four or more inlets, etc.).

FIG. 14 is a cross-sectional view of a collection device 500 according to another exemplary embodiment. Collection device 500 includes two sets of inlets such that three inlets 523, 525, and 527 that define three separate openings 524, 526, and 528 are provided on a first level and a second set of inlets 543, 545, and 547 that define three separate openings 554, 556, and 558. Openings 524, 526, and 528 taper to define openings 532, 534, and 536, and openings 554, 556, and 558 taper to define openings 582, 584, and 588. Various plates or slides 560, 562, 564, 570, 572, and 574 are associated with each of the inlets. An outlet 642 defining an opening 644 is also provided in collection device 500.

While collection device is shown as having a separate plate associated with each of the inlets, the inlets on the same level (e.g., inlets 523, 525, and 527) may share a single plate such that matter from each of the inlets impacts a different point on each of the plates. Additionally, while FIG. 14 illustrates an embodiment in which three inlets are provided on each of two levels, a different number of inlets and/or a different number of levels may be provided (e.g., a first level having three inlets, a second level having two inlets, and a third level having one inlet) for a particular collection device. The sizes, shapes, and configurations of the various inlets may be identical, or one or more of the inlets may differ in size, shape, or configuration from the other inlets. Different collection mediums may be used on one or more of the plates or slides. Any of a variety of different sizes, shapes, and configurations may be used for the various collection devices shown in FIGS. 12-14.

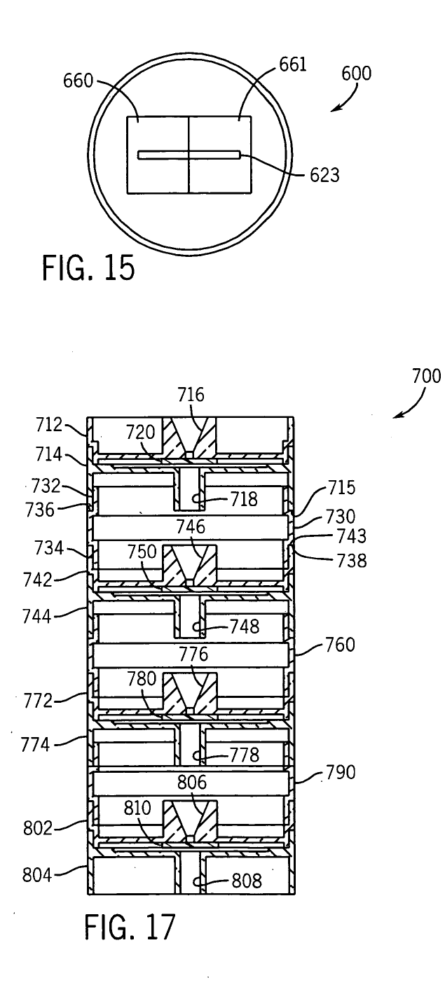

While the various FIGURES illustrate configurations in which a single slide or plate is associated with each inlet, according to various other exemplary embodiments, one or more of the inlets provided in a collection device may include multiple (e.g., two or more) associated plates or slides. For example, according to an exemplary embodiment, a collection device such as that shown in FIG. 1 may include two slides that share the gas drawn into the collection device through the inlet. FIG. 15 shows another exemplary embodiment in which a collection device 600 includes an opening 623 of an inlet that is provided above a plate or slide having two portions 660, 661 that are separated by a line scored in a surface of the plate. Gas-borne matter drawn through the inlet impacts both portions 660 and 661. After sampling is completed, portions 660 and 661 may be separated by breaking the slide along the scored line. Separate analyses may then be performed on the matter impacted on each of the two portions 660 and 661.

The matter impacted on each of the plates may be tested separately (e.g., PCR testing may be performed on the sample associated with plate 660, while ELISA testing may be performed on the sample associated with plate 661). According to another exemplary embodiment, the sample associated with one of the plates may be tested immediately after testing, while the sample associated with the other plate may be stored for subsequent testing (using either the same test or a different test).

While described with reference to a collection device similar to that shown in FIG. 1, it should be noted that any of the embodiments disclosed herein may be modified such as shown in FIG. 15 to utilize two or more plates or slides for each inlet such that gas-borne matter drawn through the inlet impacts a plurality of separate plates or slides.

It should also be noted that any of the collection devices shown or described herein may be used in conjunction with one or more filters. For example, a pre-filter may be coupled to one or more of the inlets to filter out particular types of matter before the gas enters the collection device. A post-filter may be coupled to the outlet to filter out matter after the gas has passed through the collection device.

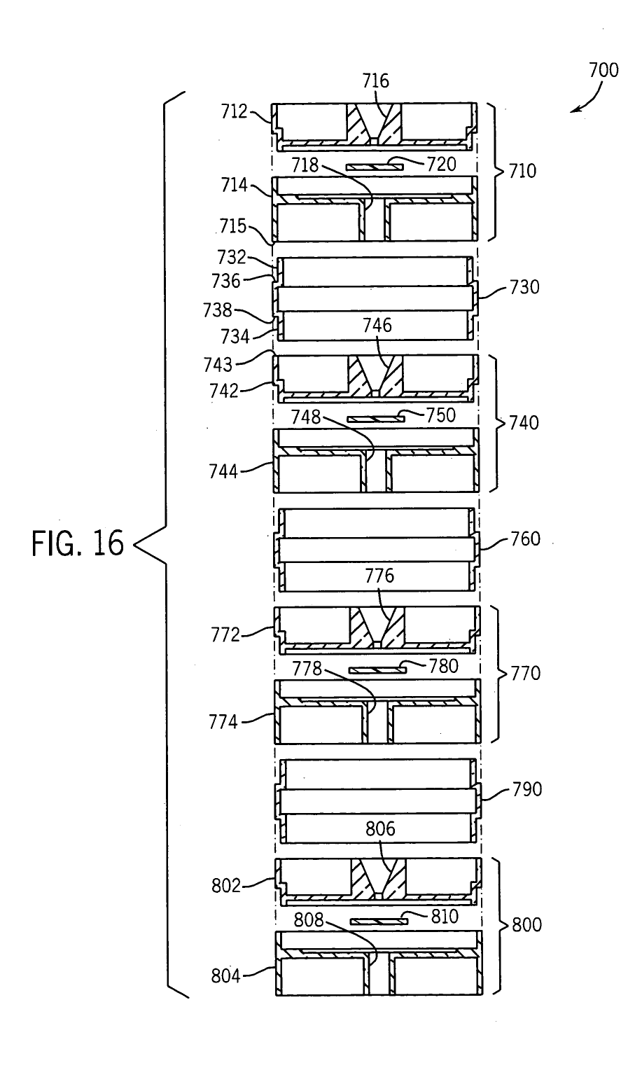

According to other exemplary embodiments shown by way of example in FIGS. 16-17, two or more collection devices may be coupled together in series. FIG. 16 shows an exploded cross-sectional view of an assembly 700 that includes four collection devices 710, 740, 770, and 800. Each of collection devices 710, 740, 770, and 800 includes a top portion (712, 742, 772, 802) having an inlet (716, 746, 776, 806) provided therein, a bottom portion (714, 744, 774, 804) having an outlet (718, 748, 778, 808), and a slide or plate (720, 750, 780, 810). Collection devices 710, 740, 770, and 800 are shown as being similar to collection device 100 shown in FIGS. 1-6, although any of the collection devices shown and described herein may be coupled together as will be described (e.g., each collection device may have multiple inlets and/or outlets, one or more of the inlets of one or more of the collection devices may be used with more than one plate, a plurality of inlets of one or more of the collection devices may be used with a single plate, etc.). It should also be noted that each of the collection devices coupled together may be identical or may differ from one another (e.g., the sizes, shapes, and/or configurations of the inlets for the various collection devices may be identical or one or more of the inlets may differ from the others).

A member or element 730, 760, 790 in the form of a coupling or connector (e.g., a connecting sleeve, collar, adapter, etc.) may be used to connect or couple the various collection devices 710, 740, 770, 800 together. For example, coupling 730 is shown as having a first portion 732 for insertion into a portion of bottom portion 714 of collection device 710 and a second portion 734 for insertion into a portion of top portion 742 of collection device 740. The bottom 715 of bottom portion 714 engages a ledge 736 formed in coupling 730 when bottom portion 714 is secured to coupling 730; the top 743 of top portion 742 engages a ledge 738 formed in coupling 730 when top portion 742 is secured to coupling 730. Each of the other couplings and collection devices may have similar features to allow coupling of the various components together in the finished assembly 700.

FIG. 17 illustrates the assembly 700 having the various components thereof coupled together such that the various collection devices 710, 740, 770, and 800 are arranged in a “stacked” or series arrangement. According to an exemplary embodiment, the collection devices are coupled together by coupling a first collection device to a first coupling and a second collection device to the first coupling. One or both of the first collection device and the second collection device may then be coupled to a second coupling, which may then be coupled to a third collection device. This process may be repeated until the desired number and arrangement of collection devices is obtained.

The couplings and collection devices are configured such that they may be relatively securely fastened together (e.g., friction prevents the components from being too easily decoupled from one another) according to an exemplary embodiment. Further, while FIGS. 16-17 illustrate an assembly having four collection devices coupled together, a different number of collection devices (e.g., two to three or greater than four collection devices) may be coupled together according to various other exemplary embodiments.

One advantageous feature of providing couplings to connect a plurality of collection devices together is that the gas sampling may be customized to the particular application in a relatively simple and efficient manner. For example, according to an exemplary embodiment, collection device 710 may be configured (e.g., size/shape/configuration of inlet, collection medium, etc.) for collection of mold spores or other relatively large particles. Collection device 740 may be configured for collection of bacteria or other matter that has a smaller size than those collected by collection device 710. Collection device 770 may be configured for collection of viruses or other matter that has a smaller size than those collected by collection device 740. Collection device 800 may be configured for collection of nanoparticles or other matter that has a smaller size than those collected by collection device 770. According to other exemplary embodiments, any suitable combination of collection devices may be utilized such that their use during gas sampling is tailored for a particular sampling application.

While the couplings 730, 760, 790 are shown as being configured for insertion into the various top portions and bottom portions of the collection devices, other configurations may also be used. According to an exemplary embodiment, features (e.g., dimples, ribs, flanges, etc.) may be formed in the couplings and in the collection devices to allow the couplings to engage the collection devices to secure a plurality of collection devices together. According to another exemplary embodiment, a portion of a coupling may be configured for insertion into a portion of a collection device (e.g., a “male” type configuration), while a second portion the coupling may be configured for receipt within a portion of a collection device (e.g., a “female” type configuration). According to an exemplary embodiment, both ends of the couplings may be provided as “female” type connectors. One of ordinary skill in the art reviewing this disclosure will recognize that a variety of configurations may be used for the couplings to allow coupling a plurality of collection devices together, and each of these configurations is intended to be within the scope of the present disclosure.

Various modifications may be made to the collection devices shown and described herein that may allow for optimization for a particular use. For example, the size of the opening at the bottom portion the inlets may be changed depending on the size and/or type of materials being sampled. In applications in which mold spores will be sampled, for example, the opening may have a length of approximately 0.6 inches and a width of approximately 0.030 inches. For a collection device that will be used in the collection of nanoparticles, the width of the bottom portion the inlet may be between approximately 0.001 and 0.005 inches. Virus sampling may utilize a width of the bottom portion the inlet may be between approximately 0.005 and 0.020 inches. Bacteria sampling may utilize a width of the bottom portion the inlet may be between approximately 0.015 and 0.025 inches. According to other exemplary embodiments, the size of the bottom portion of the inlet may be optimized for the particular use and may have a different shape and/or size.

The construction and arrangement of the collection device as shown in the various exemplary embodiments is illustrative only. Although only a few embodiments of the present inventions have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited in the claims. For example, elements shown as integrally formed may be constructed of multiple parts or elements, the position of elements may be reversed or otherwise varied (e.g., a bottom portion of a collection device may be inserted into a top portion of a collection device), and the nature or number of discrete elements or positions may be altered or varied (e.g., both an inlet and an outlet may be included in either a top portion or a bottom portion of a collection device; a collection device may be provided with two or more inlets and/or outlets, etc.). Accordingly, all such modifications are intended to be included within the scope of the present invention as defined in the appended claims. The order or sequence of any process or method steps may be varied or re-sequenced according to alternative embodiments. Features described with respect to one or more of the exemplary embodiments (e.g., testing methods used with the various collection devices, side ports for introducing liquid into a collection device, etc.) may be used with other exemplary embodiments described herein. Other substitutions, modifications, changes and omissions may be made in the design, operating conditions and arrangement of the preferred and other exemplary embodiments without departing from the scope of the present inventions as expressed in the appended claims.

1. A method for sampling gas-borne matter using a collection device comprising an inlet, an outlet, and a slide provided adjacent the inlet, the method comprising:
drawing a gas through the collection device such that gas-borne matter is collected on the slide;
sealing at least one of the inlet and the outlet; and
introducing a liquid into the collection device to remove the collected gas-borne matter from the slide.

2. The method of claim 1, further comprising removing at least a portion of the liquid from the collection device.

3. The method of claim 2, further comprising transferring the liquid removed from the collection device to a growth medium.

4. The method of claim 2, further comprising performing an Enzyme Linked Immunosorbent Assay (ELISA) analysis using at least a portion of the liquid removed from the collection device.

5. The method of claim 2, further comprising performing a Polymerase Chain Reaction (PCR) analysis using at least a portion of the liquid removed from the collection device.

6. The method of claim 1, further comprising performing a fluorometric analysis using at least a portion of the liquid.

7. The method of claim 1, wherein the liquid is selected from the group consisting of water, peptone water, mineral oil, a surfactant, and combinations thereof.

8. The method of claim 7, wherein the step of introducing a liquid into the collection device comprises introducing between approximately 0.25 and 1.0 milliliters of liquid into the collection device.

9. The method of claim 1, wherein the slide includes a substance for capturing the gas-borne matter.

10. The method of claim 9, wherein the substance is configured to maintain viable matter in a living state without promoting substantial growth of the viable matter.

11. The method of claim 9, wherein the substance is soluble in the liquid introduced into the sampling device.

12. The method of claim 11, further comprising dissolving the substance in the liquid.

13. The method of claim 1, further comprising freezing at least a portion of the liquid.

14. The method of claim 1, wherein the step of sealing at least one of the inlet and the outlet comprises inserting a portion of a barrier into the at least one of the inlet and the outlet to provide a watertight seal.

15. The method of claim 1, further comprising shaking the collection device to assist in removing the collected matter from the slide.

16. The method of claim 1, further comprising utilizing a vortexer to assist in removing the collected matter from the slide.

17. The method of claim 1, wherein the step of sealing at least one of the inlet and the outlet comprises sealing the outlet and the step of introducing a, liquid into the collection device comprises introducing the liquid into the collection device through the inlet.

18. The method of claim 1, wherein the step of sealing at least one of the inlet and the outlet comprises sealing the inlet and the step of introducing a liquid into the collection device comprises introducing the liquid into the collection device through the outlet.

19. The method of claim 1, wherein the collection device further comprises an aperture that differs from the inlet and the outlet and wherein the step of introducing a liquid into the collection device comprises introducing the liquid through the aperture.

20. The method of claim 19, wherein the step of sealing at least one of the inlet and the outlet comprises sealing both the inlet and the outlet prior to the step of introducing a liquid through the aperture.

21. The method of claim 19, further comprising removing at least a portion of the liquid from the collection device through the aperture.

22. The method of claim 1, wherein the collection device includes a rim and the step of drawing a gas through the collection device comprises placing at least a portion of the slide on the rim such that at least a portion of the slide is elevated above the outlet.

23. The method of claim 22, further comprising removing the slide from the rim and placing the slide in a trough defined by the rim before the step of introducing a liquid into the collection device.

24. A method for collecting gas-borne matter comprising:
providing a sampling device comprising at least one inlet and at least one outlet for allowing the passage of a gas through the sampling device and a plate provided within the sampling device for collecting gas-borne matter;
drawing a gas through the sampling device such that the plate is impacted with gas-borne matter;
blocking at least one of the inlet and the outlet after the step of drawing a gas through the sampling device;
introducing a liquid into the sampling device after blocking at least one of the inlet and the outlet; and
removing at least a portion of the liquid from the sampling device.

25. The method of claim 24, further comprising performing an Enzyme Linked Immunosorbent Assay (ELISA) analysis using at least a portion of the liquid removed from the sampling device.

26. The method of claim 24, further comprising performing a Polymerase Chain Reaction (PCR) analysis using at least a portion of the liquid removed from the sampling device.

27. The method of claim 24, further comprising performing a fluorometric analysis using at least a portion of the liquid.

28. The method of claim 24, wherein the liquid is selected from the group consisting of water, peptone water, mineral oil, a surfactant, and combinations thereof.

29. The method of claim 24, wherein the step of introducing a liquid into the sampling device comprises introducing between approximately 0.25 and 1.0 milliliters of liquid into the sampling device.

30. The method of claim 24, wherein the plate includes a substance for capturing the gas-borne matter that is impacted onto the plate.

31. The method of claim 30, wherein the substance is configured to maintain viable matter in a living state without promoting substantial growth of the viable matter.

32. The method of claim 30, wherein the substance is soluble in the liquid introduced into the sampling device.

33. The method of claim 32, further comprising dissolving the substance in the liquid.

34. The method of claim 24, further comprising freezing at least a portion of the liquid.

35. The method of claim 24, wherein the step of blocking at least one of the inlet and the outlet comprises inserting a portion of a barrier into the at least one of the inlet and the outlet to provide a watertight seal.

36. The method of claim 24, further comprising shaking the sampling device to assist in removing the collected matter from the plate.

37. The method of claim 24, further comprising utilizing a vortexer to assist in removing the collected matter from the plate.

38. The method of claim 24, wherein the sampling device further comprises an aperture that differs from the inlet and the outlet and wherein the step of introducing a liquid into the sampling device comprises introducing the liquid through the aperture.

39. The method of claim 38, wherein the step of sealing at least one of the inlet and the outlet comprises sealing both the inlet and the outlet prior to the step of introducing a liquid through the aperture.

40. The method of claim 38, wherein the step of removing at least a portion of the liquid comprises removing at least a portion of the liquid from the sampling device through the aperture.

41. A method for collecting gas-borne matter comprising:
providing a collection device, the collection device comprising an inlet, an outlet, and a plate provided adjacent the inlet for collecting gas-borne matter;
drawing a gas through the sampling device to collect gas-borne matter on the plate;
blocking at least one of the inlet and the outlet after the step of drawing a gas through the collection device;
introducing a liquid into the collection device after blocking at least one of the inlet and the outlet, the liquid selected from the group consisting of water, peptone water, mineral oil, a surfactant, and combinations thereof; and
analyzing the matter collected using at least a portion of the liquid introduced into the collection device.

42. The method of claim 41, wherein the step of analyzing the matter collected utilizes an Enzyme Linked Immunosorbent Assay (ELISA) analysis.

43. The method of claim 41, wherein the step of analyzing the matter collected utilizes a Polymerase Chain Reaction (PCR) analysis.

44. The method of claim 41, wherein the step of analyzing the matter collected utilizes a fluorometric analysis.

45. The method of claim 41, further comprising removing at least a portion of the liquid from the collection device before the step of analyzing the matter collected, and wherein the step of analyzing the matter collected utilizes at least a portion of the liquid removed from the collection device.

46. The method of claim 41, wherein the plate includes a substance for capturing the gas-borne matter that is impacted onto the plate.

47. The method of claim 46, wherein the substance is configured to maintain viable matter in a living state without promoting substantial growth of the viable matter.

48. The method of claim 46, further comprising dissolving the substance in the liquid.

49. The method of claim 41, further comprising freezing at least a portion of the liquid.

50. The method of claim 41, further comprising shaking the collection device to assist in removing the collected matter from the plate.

51. The method of claim 41, further comprising utilizing a vortexer to assist in removing the collected matter from the plate.

52. The method of claim 41, wherein the collection device further comprises a port for introducing the liquid into the collection device and the step of introducing the liquid into the collection device comprises introducing the liquid through the port.

53. The method of claim 52, further comprising removing at least a portion of the liquid through the port.
